(19) United States
(12) Patent Application Publication
(10) Pub. No.: US 2010/0015227 A1
(43) Pub. Date: Jan. 21, 2010

(54) DRIED ELECTRIFIED HYDROCOLLOID GELS HAVING UNIQUE STRUCTURE AND POROSITY

(76) Inventors: Amos Nussinovitch, Rehovot (IL); Ronit Zvitov, Rishon LeZion (IL)

Correspondence Address: WINSTON & STRAWN LLP;PATENT DEPARTMENT, 1700 K STREET, N.W., WASHINGTON, DC, 20006, US

(21) Appl. No.: 12/373,434

(22) PCT Filed: Jul. 15, 2007
(86) PCT No.: PCT/IL07/00888

§ 371 Date: Sep. 17, 2009

Related U.S. Application Data
(60) Provisional application No. 60/830,608, filed on Jul. 14, 2006.

(51) Int. Cl. A61K 9/10

(52) U.S. Cl. 424/484

(57) ABSTRACT

This invention discloses electrified freeze-dried hydrocolloid gels, having modified structures with improved properties, as well as methods for the preparation of these modified gels and their uses. Specifically gels modified by electrification and freeze-drying undergo changes including creation of concentric layers of gel and intervening spaces.

### FIELD OF THE INVENTION

This invention discloses electrified freeze-dried hydrocolloid gels, having modified structures with improved properties, as well as methods for the preparation of these modified gels and their uses. Gels modified by electrification and freeze-drying undergo changes including generation of concentric layers and increased surface area per volume.

### BACKGROUND OF THE INVENTION

Gels have been found to be useful for serving as carriers for and/or entrapping ingredients such as microorganisms, e.g., important bacteria for antibiotic production, cheese formation, or the continuous fermentation of champagne, oils, vitamins, essential nutrients, (Eskin, 1990), and other biotechnological applications (Nussinovitch, 0.1994, 1997; Nussinovitch, Nussinovitch, Shapira, & Gershon, 1994), use as mini-reactors in which synthesis or decomposition occurs (Tampion & Tampion, 1987), use in different environments, e.g. immersed in liquids (Tal, Van Rijn, & Nussinovitch, 1997, 1999), embedded in solid wet and dry porous substances (e.g. different soils for biological control of root diseases), contained in gas reservoirs or in a receptacle which allows gas exchange through their pores, and on their surfaces for decomposing toxic substances (Tal et al., 1999); or, medically, for transplantation, e.g. of beads under the skin for the slow release of drugs.

Gel properties which may be manipulated in order to increase the efficacy of gels in these uses include size, volume, surface area ratio, porosity, strength, elasticity, swelling ability, proclivity to decomposition or the ability to remain intact under different conditions of, for example, pH, acidity, osmotic pressure, presence of sequestering agents, etc. To accomplish this, swelling-shrinkage techniques can be used (Tanaka, 1981, 1992). Other processes are designed to induce changes in the polymer network structure and are affected by pH, ions, UV light, electrical fields and solvent composition (Tanaka, 1981; Tanaka, Nishio, Sun, & Uneo-Nishio, 1982). With respect to networks contracted by electrical fields, most reports have dealt with synthetic gels (De Rossi, Suzuki, Osada, & Morasso, 1992; Gong, Komatsu, Nitta, & Osada, 1997; Kishi & Osada, 1989; Shiga & Kurauchi, 1990).

U.S. Pat. No. 6,297,033, to one of the inventors of the present invention and co-workers, discloses permeable polymeric beads which contain a combination of fermentative and denitrifying bacteria and a carbon source, for use in a system for nitrate removal from aquariums. The carbon source used is preferably potato starch, and is not disclosed as imparting any structural, or mechanical properties to the beads.

U.S. Pat. No. 6,589,328 to Nussinovitch, discloses hydrocolloid sponges produced by preparing a gel of a hydrocolloid, and either sealing it in a closed vessel with a liquid of similar composition, pressurizing the vessel and abruptly releasing the pressure, followed by freeze drying, or by incorporating in such a gel a suitable microorganism, such as a yeast and inducing fermentation in the presence of a suitable nutrient medium, so that the carbon dioxide formed results in the expansion and foam formation, which is processed to the final product.

Zohar-Perez et al. (20) disclose irregular textural features of dried alginate-filler beads, having up to 0.5% (w/w) of bentonite or kaolin as fillers. These beads are further reported to provide extra protection for microorganisms against UV radiation (24).

US Patent Application Publication Number 2003/0224022 to Nussinovitch disclosed hydrocolloid cellular solid matrices that are useful as carriers for a variety of substances.

Electrically induced changes, including shrinkage, of gels in different fluids and/or electrical fields have been investigated as a means of improving their structural and mechanical properties, such as porosity. However, most of the moieties examined under these conditions have consisted of polyacrylamide gels in water/acetone combinations (Tanaka, Nishio, Sun, and Uneo-Nishio 1982).

Electrically induced changes of hydrocolloid gels other than polyacrylamide and changes in their shape, porosity, mechanical properties and chemical changes caused by the electrical treatment were reported by these inventors. The behavior of a few types of hydrocolloid gels under the application of a low electrical field has been discussed in previous reports by the inventors (Zvitov and Nussinovitch 2001; Zvitov and Nussinovitch 2003; Zvitov, Zohar-Perez, and Nussinovitch 2004). One of the electrical treatment's benefits was the production of pores at the surface of the treated specimen, which could change its release properties for special applications. The inventors also reported that, with regard to gel beads, agarose appears to be less affected by the DC electrical application (some small changes at the surface) than alginate gel beads; in both gels, however, the shape of the affected area of the shrunken specimen resembled the shape of the anode (Zvitov and Nussinovitch 2003).

Zvitov and Nussinovitch (2001) discloses weight, mechanical and structural changes induced in alginate gel beads by electrification. This publication does not disclose the use of freeze-drying, and merely speculates about the possible outcome combinations of electrification and freeze drying. Zvitov et al (2003) discloses changes induced by DC electrical field in agar, agarose, alginate and gellan gel beads. Additionally, US 2006/0254912 to Nussinovitch discloses a method for treating biological organic tissue, particularly plant tissue, by applying a direct current for extraction and separation of substances of interest from the biological tissue. The materials that are subjected to electrification are not gels, the methods do not include freeze-drying.

There thus remains an unmet need for gels possessing improved structural and mechanical properties, such as modified porosity, and more efficient and effective methods of obtaining those structures and properties.

### SUMMARY OF THE INVENTION

The present invention relates to hydrocolloid gels having specific modified structures with improved properties induced by electrical treatment combined with freeze dehydration. The improved properties of the gels result from changes in their structural and mechanical properties.

It is now disclosed for the first time that electrification of hydrocolloid gels followed by freeze drying induces a novel structure of concentric layers of the gel. The concentric layers can be induced to varying extent depending on the type of gel and the procedures used. Concentric layers of the hydrocolloid material provide increased surface area of the gel material within the freeze-dried product compared to gels that have not undergone this treatment.

According to one aspect of the present invention, electrified freeze dried gels having concentric layers of hydrocolloid gel material, separated by intervening spaces are disclosed. The novel features induced in the structure of the hydrocolloid gels by the combination of electrification, followed by freezing and drying may be advantageous in terms of selected properties including porosity, density or size of pores, volume, surface area ratio, strength, elasticity, swelling ability, proclivity to decomposition or the ability to remain intact under different conditions. As is known in the art additional factors may influence the gel properties prior to electrification and freeze drying including among others pH, osmotic pressure, presence of sequestering agents, and presence of other active or inert agents.

Parameters of the gel itself, such as the type of gel, the means by which it is cross-linked, its pH and even its shape, may influence the changes induced by electrification and subsequent freeze-drying.

Gels provided according to the present invention may be obtained in all forms and shapes, including, but not limited to, beads, plates, strips, sheets and cylinders.

According to a first aspect of the present invention, electrified freeze-dried gels having concentric layers of hydrocolloid gel material, separated by intervening spaces are disclosed. According to the principles of the present invention, concentric gel layers separated by intervening spaces created by the electrification followed by freezing and drying impart at least one improved property of the gels. The concentric spaces according to some embodiments are generally parallel to the circumference of the gel. According to a specific embodiment the concentric spaces are not visible at the outer surfaces of the gels but are present internally and may be seen in cross-sectional analysis (e.g., by viewing cut beads, even if only a thin layer has been removed).

According to specific embodiments hydropolymers suitable in the context of the present invention are selected from agar, agarose, pectin, carrageenan, alginate, and low methoxy pectin. Other gelling agents such as chitin, chitosan, curdlan, konjac and combinations thereof can also be used for the gellification and bead formation. According to certain embodiments the gel is selected from the group consisting of alginate, agarose and Low Methoxy Pectin (LMP) gel. According to a specific embodiment the gel is alginate.

According to one embodiment the at least one improved property is a higher overall surface area of the gel material within the final structure. In other words the surface are of the gel within a given volume of the gel product is significantly higher than the surface area of the gel having the same composition that was not electrified. This may be advantageous in fields such as delivery of an active agent, water denitrification, biotechnology and food preparation.

The present invention provides, in another aspect, uses of gels having improved properties due to the existence of concentric layers and intervening spaces. According to some embodiments the gels may be used in the food industry, for example, as carriers for food snacks. According to other embodiments the gels may be used in biotechnological processes, for example, for entrapping ingredients such as microorganisms, or for use as mini-reactors in which synthesis or decomposition occurs. According to another embodiment medical uses of the gels are provided for drug entrapment and delivery options, for example, as carriers allowing for modified release of drugs. According to yet additional embodiments, the gels may be used in agriculture, for example, in biological control of plant diseases. An additional embodiment according to the present invention includes use of the gels for decomposing toxic substances. According to this embodiment gels may be used in different environments, e.g., immersed in liquids, embedded in solid, wet and dry porous substances, contained in gas reservoirs or in a receptacle which allows gas exchange through its pores, and on their surfaces.

According to another aspect the present invention provides a method for preparing a gel composition having improved properties, the method comprising electrification and freeze-dehydration. According to some embodiments the freeze-dehydration follows the electrification of the gels results in changes in structure and shape to the dried cellular solids. These changes, which were not previously disclosed or suggested, do not occur in the absence of the freeze-drying step. According to some embodiments the method induces the formation of concentric layers within the gel structure. According to some specific embodiments the structural features may include the creation of enlarged porosity and decorative structures on the gels.

According to a specific embodiment the method for preparing a gel having improved properties comprises the following steps:
a) providing a gel specimen;
b) electrification of the gel specimen by applying a DC voltage;
c) freezing the gel specimen; and
d) freeze-dehydration of the gel,
   thereby changing at least one of its properties.

The method for preparing a gel having improved properties according to the present invention is applicable to all forms and shapes of gels. According to a specific embodiment, the gel is provided in beads form. According to another embodiment the gel is provided in a form other than beads.

According to one embodiment the DC voltage applied in step b) is ranging from 0.1-40 V at electrical field strength up to 40 V/cm. In the event that the gels are formed as films or sheets higher voltages may be applied. According to a specific embodiment the electrification is performed using an apparatus described in WO 2004/078253. According to one embodiment, the at least one property changed is creation of concentric layers and spaces within the gel treated. According to another embodiment the at least one property changed is increased porosity. According to a specific embodiment the concentric spaces are created at the cathode end of the gel, namely at the side of the gel closer to the cathode during electrification. According to yet another embodiment the texture of the gel is changed as a result of the method applied.

According to one embodiment an ion solution may be added to the gel before electrification performed. According to a specific embodiment the ion solution is CaCl₂ or BaCl₂. According to another embodiment the pH of the gel is modified before performing the electrification step.

According to the methods of the present invention, different gel types, comprising different ingredients, are affected differently when subjected to said methods. Specifically, the extent to which properties such as porosity, size, volume, surface area ratio, strength, elasticity, swelling ability, proclivity to decomposition or the ability to remain intact under different conditions are changed is dependent on the type of gel to which the method is applied. Specific factors that may influence the outcome or the extent of the changes induced by electrification and freezing may include pH, acidity, osmotic pressure, presence of sequestering agents, presence of active or inert ingredients and the like.

Another aspect of the present invention provides pharmaceutical compositions comprising gels according to the present invention and at least one therapeutic agent. The therapeutic agents include according to one embodiment growth factors, cytolines, chemotherapeutic drugs, enzymes, anti-microbials, anti-resorptive agents and anti-inflammatory agents and essential oils. The pharmaceutical compositions may further comprise a pharmaceutically acceptable carrier or excipient.

The pharmaceutical composition can be dispensed in many different forms, depending on the indication and discretion of the medical practitioner. In some embodiments the composition is a dry composition, for example particles, granules or powder, optionally obtained by lyophilization. In certain indications a fluid, or semi-fluid composition is provided.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows SEM micrographs of freeze-dried alginate specimens: A-B) whole specimen and magnification, respectively; C-D) electrically treated specimen from the anode side and magnification, respectively; E-F) electrically treated specimen from the cathode side and magnification, respectively, in accordance with some embodiments of the present invention;

FIG. 2 shows SEM micrographs of freeze-dried agarose specimens: A-B) whole specimen and magnification, respectively; C-D) electrically treated specimen from the anode side and magnification, respectively; E-F) electrically treated specimen from the cathode side and magnification, respectively, in accordance with some embodiments of the present invention;

FIG. 3 shows the stress-strain relationship of agarose and alginate dried specimens before and after electrical treatment (10 V/cm, 1 min), in accordance with some embodiments of the present invention;

FIG. 4 shows the number of pores/specimen vs. pore size (mm²) as derived by image analysis for alginate and agarose dried specimens before and after electrical treatment, in accordance with some embodiments of the present invention;

FIG. 5 shows SEM micrographs of freeze-dried alginate beads: A) whole bead; B) electrically treated whole bead from the cathode side; C) cut bead; D) electrically treated cut bead from the cathode side, in accordance with some embodiments of the present invention;

FIG. 6 shows SEM micrographs of freeze-dried alginate specimens: A, C, E) outer surface; B, D, F) cut surface. A-B) Whole specimens; C-D) electrically treated specimens from the anode side; E-F) electrically treated specimens from the cathode side, in accordance with some embodiments of the present invention;

FIG. 7 shows SEM micrographs of freeze-dried alginate (A-C) and agarose (D) specimens: A) electrically treated specimen from the cathode side; B) alkaline-treated specimen; C) control; D) agarose immersed in CaCl2 prior to the electrical treatment, from the cathode side, in accordance with some embodiments of the present invention;

FIG. 8 shows SEM micrographs of freeze-dried alginate specimens: A, C, G) whole specimens, no electrical treatment; B, D, H) specimens after electrical treatment from the cathode side. A-B) Specimens immersed in distilled water for 24 h; C-D) alginate specimens cross-linked with BaCl2; G-H) alginate gel specimens produced by cold-set procedure. E-F) SEM micrographs of freeze-dried gellan specimens, before and after electrification, respectively, in accordance with some embodiments of the present invention;

FIG. 9 shows SEM micrographs of freeze-dried LMP specimens: A) whole specimen; B) electrically treated specimen from the cathode side, in accordance with some embodiments of the present invention;

FIG. 10 shows SEM micrographs of freeze-dried alginate specimens cross-linked from the top: A) whole specimen; B) electrically treated specimen, in accordance with some embodiments of the present invention;

FIG. 11 shows SEM micrographs of alginate gel specimens after freezing and thawing cycle: A) whole specimen; B) electrically treated specimen, in accordance with some embodiments of the present invention;

FIG. 12 is a graph of OD535 values versus time for untreated (control) and electrically treated alginate specimens immersed in distilled water, in accordance with some embodiments of the present invention.

### DETAILED DESCRIPTION OF THE INVENTION

The present invention relates to hydrocolloid gels and beads made from these gels having improved properties imparted by electrification combined with subsequent freezing. These gels and these beads and other products formed of the gels, are useful per se or serve for carrying active agents.

The beads of the present invention have unique attributes in terms of shape, surface structure and porosity obtained by exposing the gels to electrification and subsequent freezing, preferably freeze-drying. The beads so obtained have concentric layers of gel thereby creating a concentric series of intervening spaces. These structures have never been observed previously and are now shown to possess advantageous properties in terms of drug loading capacity and use as traps for active agents of choice.

The term “bead” in the context of this invention refers to particulate material, having any desired geometric shape, or a shape selected from a spherical shape, a sphenoid shape, an ellipsoid shape, a teardrop-like shape, an oblong shape and a rectangular shape.

According to some embodiments, the beads are formed having a size range of 100 microns to 5 cm and the size of the bead can be tailored according to the specific need. For many applications the average diameter of the beads will be conveniently in the range of several millimeters to several centimeters typically 1-20 mm. It will be appreciated that after electrification the beads undergo shrinkage compared to the beads prior to electrification and addition the beads may further shrink upon drying

Especially small carrier particles may be obtained by spray drying or by using selected size reduction equipment such as ball mill, roller mills, pin and disc mill, and the like. Larger beads in the range of 1 cm may be achieved by dropping and solidification. It is necessary to distinguish between the sizes of the original gel matrices or gel particles and the dried particles. The skilled artisan will appreciate that it is easier to apply the methods of the present invention to particles or objects having a larger dimension. Thus, it is more convenient to apply the electrification step to a body having suitable dimensions. This is correct for gels of any shape. After electrification and drying it is possible to reduce the size of the product to the desired size. For example the dried moieties being more porous and having the novel structure of the invention can be ground or milled if appropriate in order to achieve smaller particles.

The term “hydrocolloid material” refers to a hydrocolloid, a gum, or a gum-resin being a water soluble polymer which, in the presence of an aqueous medium, forms a hydrocolloid gel upon cross-linking or by hydrogen bonds. The material may be obtained from a natural source, may be a hydrocolloid from natural source that has been chemically modified, or may be synthetic. Typically the hydrocolloids are polymers and more specifically polysaccharide or polypeptides polymers. As used herein the term “hydrocolloid material” includes both polysaccharides and proteins. For example gelatin and casein are proteins that are regarded as hydrocolloids. The natural hydrocolloids may be of animal, vegetable or microbial origin. For example, agar and alginate are from algae, chitosan is derived from chitin extracted from crustaceans; and gellan is a microbial hydrocolloid.

According to some embodiments of the invention, including those using an alginate gel, it is preferable to cross-link the hydrocolloid by using bivalent cations such as Ca++, Fe++, Sr++, Pb++, Ba++, or trivalent cations such as Al+++. For gellan, other cations are used. An alternative to cross-linking is to form hydrogen bonds, which in the case of agar or agarose are produced spontaneously in the gelling process.

Non-limiting examples of hydropolymers suitable in the context of the present invention are agar, agarose, pectin, carrageenan, alginate, and low methoxy pectin (LMP).

Other gelling agents such as chitosan, curdlan, konjac, combinations of carrageenan and xanthan or carrageenan and locust bean gum (LBG) and additional combinations thereof can also be used for the gellification and bead formation. Typically, in the gels, the hydrocolloid material is 0.02 to 20% (w/w), and preferably 1 to 15% (w/w), more preferably and most preferably 1 to 3% (w/w) of the wet bead. In the case of gelatin, higher concentrations, such as 10-15% (w/w) on a wet basis, are used.

The present invention further discloses a pharmaceutical composition comprising the beads as described herein, carrying one or more active agents. The pharmaceutical composition may optionally comprise a pharmaceutically acceptable carrier.

In pharmaceutical compositions comprising “empty” beads, the beads themselves induce a therapeutic effect. For example, they may reduce the level of a compound in excess by absorption thereof from the body of a subject. Empty beads may be used, for example, to reduce cholesterol levels, to detoxify subjects and to treat drug and medicament overdoses especially in the framework of stomach pumping.

The present invention is further directed to a composition for use in agriculture comprising beads as described herein and an optional carrier.

The purpose of the composition comprising the “empty” beads is the controlled release of the bead components into the soil, for example as a source of nutrients for beneficial microorganism (fungi). Empty beads may also be used as a carbon or nitrogen source. The electrified freeze dried gels of the present invention may further be useful in water denitrification. The present invention further concerns a composition comprising the beads as described herein, loaded with at least one active agent.

Typically, a dried bead may have a shelf life of at least two years.

The term “active agent” refers to an organic or inorganic compound, a biological material, or complex of compounds that affects the ambient surrounding of the bead in a desired manner, and for which slow and controlled release is beneficial.

By one embodiment, the active agent is a medicinally active agent, such as, but not limited to, a drug, a diagnostic agent or an imaging agent.

The medicinally active agent may be any drug, pro-drug, combination of drugs, diagnostic agents, or imaging agents used in therapy or diagnosis.

The drugs used in the beads of the present invention may be drugs with an improved medicinal activity in a controlled-release profile relative to a free form. The drug may either be water soluble or insoluble.

When a hydrophobic drug is used, the carrier beads may include a small quantity of oil and/or fat for solubilization of hydrophobic drug. The bead can be tailored for carrying any possible drug or materials specified above. The carrier biological agents may be selected from proteins, antibodies, peptides, nucleic acid based compounds and microorganisms that have a beneficial effect, such as probiotic bacteria.

According to some embodiments, the active agent may be diltiazem hydrochloride in beads in which the amount of the filler is at least 10% w/w of the preparation media.

The medicament or pharmaceutical composition should preferably be adapted for oral administration although other modes of administration are construed to be within the scope of the present invention.

In the case of topical and mucosal administration, the beads may be incorporated into another matrix, such as in a patch (glue). The patch may be used to place the beads in a sustained manner on the skin or mucosal tissue, as is known in the art.

By another embodiment, the active agent may be an agriculturally active agent such as an agro-chemical compound used for control of pests, as a herbicide, a fertilizer, a biological compound, or active microorganism used for biological control of pests and disease. In such a case, the composition may further comprise carrier acceptable for agricultural use.

The “agriculturally active agent” may be any organic, inorganic or biological agent used in agriculture. This also includes biological agents, such as live agents, including microorganisms, for control of pests, as a herbicide, as a fertilizer, for supplying vitamins, minerals, pigments and preservatives to an agricultural environment. Some embodiments relate to the use in agriculture in the biological control of pests and disease, such as for biological control of root diseases etc to be applied to the soil, to a plant or to an aquatic environment, such as a pond, river or sea.

It should be noted that the requirements of an agriculturally active composition are different from a pharmaceutical composition. First, some harsh conditions that characterize the environment of drugs especially orally administered drugs, such as low pH, do not characterize compounds administered to soil, plants or water. On the other hand, while the beads in the body are typically exposed to a constant moist environment, beads used for agricultural purposes are many times exposed to drastically varying moisture contents and can revert from dry to wet stages.

In addition, the composition applied to soil is exposed to varying temperatures and to UV irradiation, as well as exposure to microorganisms in soil, such as bacteria and molds, which are very different to the bacterial flora of the gastro-intestinal (GI) tract.

Due to the different localized environments in which the beads are to release one or more active agents, the beads need to be designed differently according to their end application. Thus, compositions that are used for agriculture have to be tailored differently to those used in therapy.

By yet another embodiment, the active agent may be an agent used in the food industry or in the preparation of nutraceuticals such as vitamins, preservatives, pigments, taste enhancing compounds and functional food components

According to yet another embodiment, the active agent may be a chemical, an enzyme, a reagent, a starting material for use in industry in chemical or biochemical reactions.

The present invention further concerns a method for the preparation of the above bead the method comprising:
a) providing hydrogel material dissolved in aqueous media;
b) forming beads;
c) electrifying the beads;
d) freezing the beads; and
e) drying the beads

Optionally the process will further comprise introducing an active agent into the beads, either before electrifying, before freezing, before drying or after drying. For many active agents the agent will be added to the dried beads It will be appreciated by the artisan that adding the active agent prior to electrification is possible only if the active agent can withstand the electrification step, e.g., if it is thermostable. There are pharmaceutical ingredients, agricultural agents, foodstuffs and even microorganisms that are thermostable.

It should be noted that the duration of electrification can be selected to suit the protocol used. Thus, in certain embodiments the electrification can be very short, i.e. a few seconds. Even if reduction in the active ingredients or microorganism number does occur, these losses may be acceptable depending on the actual agent used in the bead. According to some alternative embodiments, it is also possible to add the active agent by spraying the dried gels of the invention with an active agent with or without additional coating. According to some embodiments it is further possible to include one or more active agent within the gels and to coat the external surface with an additional layer of the same or another active ingredient.

It is important to note that electrification can take place in liquid or out of liquid when the outer surface of the gel is wet to conduct the electric current.

The present invention provides, in another aspect, uses of gels having improved properties due to the existence of concentric layers and intervening spaces. According to some embodiments the gels may be used in the food industry, for example, as carriers for food snacks. According to other embodiments the gels may be used in biotechnological processes, for example, for entrapping ingredients such as microorganisms, or for use as mini-reactors in which synthesis or decomposition occurs. According to another embodiment medical uses of the gels are provided for drug entrapment and delivery options, for example, as carriers allowing for modified release of active agents, including drugs. According to yet additional embodiments, the gels may be used in agriculture, for example, in biological control of plant diseases. Additional embodiment according to the present invention includes use of the gels for decomposing toxic substances. According to this embodiment gels may be used in different environments, e.g., immersed in liquids, embedded in solid, wet and dry porous substances, contained in gas reservoirs or in a receptacle which allows gas exchange through its pores, and on their surfaces.

Step (b) “forming beads” may be achieved in at least two ways. Typically when beads are formed by hydrogen bond formation, the media may be dropped into an appropriate fluid, such as cold water or mineral oil. In a particular embodiment the molten polymer may be dropped through a thin oil layer into the hydrophilic medium (such as water, salt solution, etc), e.g. in the case of agar/agarose. However, when beads are formed by cross-linking, the formation occurs by dropping the solution/suspension/dispersion/emulsion of (a) into a bead forming (cross-linking) solution, or by spraying.

The “bead forming” solution may be a cross-linking solution which is in excess (for example when using alginate, gellan or chitosan) for producing a particulate bead.

By another option the “bead forming solution” may have a cross-linking agent solution having an oil layer floating above it, which helps form the beads such as in cases where agar or agarose is used.

The drying option depends on the application. If microorganisms are embedded, the drying may be performed by any of the methods set forth herein below. The particular method employed depends on the amount of residual moisture intended to be maintained in the bead, the condensation of the bead (higher temperature produce more condensed beads) the nature and sensitivity of the active material, the size of the desired bead.

Drying can be performed by a method selected from: vacuum drying, freeze drying, spray drying, fluidized bed drying, oven drying, solar drying, infra-red drying and electrical drying. However, the higher the drying temperature, the higher is the resultant density of the bead. If empty beads are prepared, the drying temperature is less critical as there is no active agent therein. Empty beads may be used per se for medical applications e.g., for absorbing cholesterol, drugs, and toxins, and for agricultural purposes e.g. for absorbing especially fertilizers.

According to currently preferred embodiments the drying step is suitably freeze drying whereby the freezing step and the drying step are carried out in a single procedure.

In order to change the dimensions and the bulk density of a preparation, a compression step may be included.

The present invention is further directed to a method for preparation of the above composition including an active agent being a drug, an agent used in food industry, an agent used in agriculture.

The active agent can be added to the hydrocolloid solution in step (a) or to the dried beads after they are formed. It is also possible to load it through diffusion into the bead from the outside by placing them in a medium containing the desired active agent. According to some embodiments a large excess of ingredient is included in the fluid medium in which electrification takes place. Thus both changes in surface area and inclusion of ingredient occur in the same step at the same time.

By another alternative, where the active ingredient (drug, agent used in agriculture) is hydrophobic, the active agent can be included in a fatty material that is inserted into the bead by infusion (placing the beads in the media comprising the active material preferably under vacuum), the fatty material comprising the active agent inside and/or on the bead is then allowed to solidify.

Another option is by spraying sticky powder containing the active ingredient on the formed bead (or powder on a sticky surface), or trying to force it “as is” through the open pores of the bead under pressure.

In a forming hydrogel solution step, water, at least one polymer, and other materials are mixed together. In some cases, one or more active agents are added to the solution in this step.

Typically the ratio of the at least one polymer to the water is 0.5-20% (w/w). In some cases, the ratio is 1-3%, and in others 10-20%.

Water may be tap water, distilled or deionized water, depending on the application. At least one polymer may be selected from, for example, but not limited to, agar, agarose, pectin, carrageenan, alginate, gellan, konjak mannan, xanthan gum and locust bean gum (LBG), or a combination thereof. Other gelling agents may be used such as chitosan, starch, gelatin, curdlan, and combinations thereof.

Additives may be added during this step. These additives may include one or more of an emulsifier, buffer, surfactant, a pH modifying agent, stabilizer and coloring agent, as are known in the art. For agricultural applications, additional or alternative additives may be added according to the particular application.

At least one active agent may be added during this step or during ensuing steps. The term “active agent” refers to an organic or inorganic compound, a biological material, or complex of compounds that affects the target, whether in vivo or in the environment in a desired manner. According to some embodiments the active agent will be released from the beads comprising the inert filler in a slower release profile than would be obtained from the beads having the same composition without the inert filler. The carrier beads of the invention are therefore advantageous for active agents for which slow and/or controlled release is beneficial.

By one embodiment, the active agent is a medicinally active agent, such as, but not limited to, a drug, a diagnostic agent or an imaging agent.

The medicinally active agent may be any drug, pro-drug, combination of drugs, diagnostic agents, or imaging agents used in therapy or diagnosis.

Typically polymer(s), water and optional additive(s) are mixed by stirring under gentle heating (30-50° C.) to form a hydrogel solution. Agent(s) may be added under gentle heating or to the solution after cooling.

In a bead forming step, the preparative bead solution is added to a gelling solution. The ratio of these solutions is typically such that the beads produced comprise 0.02 to 20% (w/w) of the hydrocolloid/polymer material, and more preferably 1 to 15% (w/w), and most preferably 1 to 3% (w/w), together with 10 to 15% (w/w) of filler material. The beads formed typically comprise 0-3% of active agent, selected from at least one of

Gelling solution typically comprises bivalent cations such as Ca++, Fe++, Sr++, Pb++, Ba++, or trivalent cations such as Al+++. In some cases univalent ions such as K+ may be used for gelling of kappa-carrageenan. There may be several sub-steps to this step. For example, salts containing the bi/tri-valent ions may be dissolved at a temperature range of 60-100° C. and the resultant solution may be cooled to 50° C. Thereafter, the bead preparative solution may be added to the resultant solution. Many alternatives to these sub-steps are construed to be within the scope of this invention. In this regard, see the examples herein below.

An alternative to cross-linking the polymer is to form the beads using hydrogen bonds which in the case of agar or agarose are produced spontaneously in the gelling process.

Gelling solution may optionally comprise one or more additional gelling agents such as chitosan, starch, gelatin, curdlan, konjac mannan in the bead formation step.

According to some embodiments, at least one active agent may be added at this stage. As noted above the active agent can be added at each step, and the selection of the most beneficial step will be made depending on the choice of the active agent.

Typically in the hydropolymer bead preparative solution, the filler material comprises 10 to 15% (w/w) thereof (which translates to around 50 to 70% (w/w) of the dried weight of the bead).

The drugs used in the beads of the present invention may be drugs with an improved medicinal activity in a controlled-release profile relative to a free form. The drug may either be water soluble or insoluble.

When a hydrophobic drug or water insoluble drug is used, the carrier beads may include a small quantity of oil and/or fat for solubilization of hydrophobic drug. or an emulsion containing same. It might be possible to emulsify the hydrophobic material within the gelling solution if an emulsifier is present. The bead may be tailored for carrying any possible drug or materials specified herein. The biological agents may include proteins, antibodies, peptides, nucleic acid based compounds and microorganisms which have a beneficial effect, such as probiotic bacteria.

In this forming beads step, the method of mixing preparative solution with the gelling solution will determine the wet bead size and physical/chemical characteristics thereof. For example, if solution is dropped into gelling solution, the size of the drops will largely determine the size of wet beads formed therefrom.

It should be understood, that in certain examples, no active agent is added in any of steps and the beads thus formed will be empty beads. These beads may be used in medicine, agriculture or in environmental engineering to absorb poisons, toxins or other chemicals from a body, from the soil, from an aquatic or gaseous environment, respectively.

Typically, in the beads of the present invention, contain 0.02 to 20% (w/w) of the hydrocolloid/polymer material, and more preferably 1 to 15% (w/w), and most preferably 1 to 3. % (w/w) of the wet bead. It should be noted that while for most hydrocolloids, 1-3% w/w of the bead is the preferable range, for gelatin the preferable range is 15-20% w/w.

According to currently preferred embodiments the gel is selected from agarose, alginate and low methoxy pectin. Specific differences in the structure were observed when using any particular gel substance

Before drying, a steeper pH gradient (˜2 near the anode and 12 near the cathode) is observed in the alginate gels. Agarose gels yield pH values similar to those of the alginate gels if they include CaCl₂ added by diffusion, but no spaces are produced on their outer surface. Alginate gels that contain no extra ions (having previously diffused out) do not produce surface pores after electrification. pH is another factor involved in the formation of the new structures. Low Methoxy Pectin (LMP) gels resemble alginate in their cross-linking mechanism, and produce similar shapes upon electrification. If gels (alginate or LMP) are manufactured in a cubic shape, the created spaces are parallel to the rectangular base and run along the prism's axis. When gels are electrified to cause small changes in weight/length (up to 40 V/cm) but are still far from collapse, a weight reduction, imprinting of the electrode shape on the surface of the shrunken gel, mineral diffusion, changes in the treated specimens' mechanical properties and local changes in gel pH, as reported previously by these inventors together with a suggested mechanism for the observations and an identification of the resemblance between electrified plant tissue and gels has been noted by the inventors (Zvitov and Nussinovitch 2001; Zvitov, Schwartz, Zamski, and Nussinovitch 2003; Zvitov and Nussinovitch 2005). The use of relatively low electrical field strength is desirable to minimize the absorption energy of the treated systems, which could be transformed into heat.

The following examples are provided merely in order to illustrate some embodiments of the present invention and are to be construed in a non-limitative manner.

### Materials and Methods

### Hydrocolloid Gels

Agarose (Sigma Chemical Co., St. Louis, Mo.) gels are prepared by dissolving the respective gum powder (1-3%, w/w) in heated distilled water until boiling and holding it at that temperature for at least 1 min. Agarose cylindrical gels (4×3-20 mm, thickness by diameter) are obtained by cooling the gel solution to a temperature above the setting temperature of the gels (˜50° C.), before pouring them into Petri dishes. After the gels are cast, they are left to equilibrate before taking the cylindrical specimens using a cork borer. To obtain the exact height, the cylinders are cut with a novel custom-made cutting device described previously (Zvitov and Nussinovitch 2005). It is important to note that the agarose is not dialyzed and thus contains some free ions, as has been previously detailed (Zvitov and Nussinovitch 2003); in addition, for some embodiments these agarose gels are immersed in a 2% (w/w) CaCl₂ solution for 24 h.

Alginate gel cylinders are produced by placing a sodium-alginate (G:M ratio of 39:61) (Sigma) solution (2%, w/w) (Nussinovitch, Peleg, and MeyTal 1996b) in a cellulose dialysis sleeve (Membrane Filtration Products, Inc., Seguin, Tex.) and immersing the sleeve in a cross-linking solution bath (0.2 M CaCl₂ or 0.2 M BaCl₂) till gelation (24 h); the gel cylinder is cut into smaller cylinders (4×3-20 mm, thickness by diameter) utilizing the aforementioned cutting device. In addition, alginate gel cylinders are prepared by mixing the aforementioned alginate solution with 1.5% (w/w) sodium hexametaphosphate (SHMP; BDH, Poole Dorset, U.K.) for 30 min while heating to ca. 40° C., prior to the addition of 1.5% (w/w) CaHPO₄ (Riedel-de Haen, Seelze, Germany), which is incorporated for 60 min. The mixture is cooled to 20±1° C. and 3.0% (w/w) fresh glucono-δ-lactone solution (GDL, Sigma) is mixed in. The volume of the GDL solution is approximately 10% of the overall gum solution. The mixture is poured into a Petri dish and left overnight (15 h) at 4° C. for gelation. Cylinders (12×12 mm, diameter by height) are produced by cork borer. Alginate beads are produced by dropping a sodium-alginate (G:M ratio of 39:61) (Sigma) solution (1-3%) into a cross-linking solution (0.125 M CaCl₂) as described previously (Nussinovitch, Peleg, and MeyTal 1996a). Gellan cylinders are produced in the same manner as the alginate cylinders, but with gellan solution (2%, w/w) (Sigma) and the same CaCl₂ solution.

### Drying Procedure

All specimens are frozen at −80° C. for 1 h before freeze-drying, which is carried out at −50° C. at a pressure of 1.1 Pa (Martin Christ model ALFA I-5; Osterode am Harz, W. Germany) (Tal, van Rijn, and Nussinovitch 1999).

### Electrical Apparatus

A custom-made apparatus has been built to permit electrical treatment of cylindrical gels in liquid medium (Zvitov and Nussinovitch 2005). Dry gel specimens (4×6.5 mm, thickness by diameter) are sandwiched between a pair of platinum electrodes (Holland Moran LTD., Yehud, Israel) and the space is filled with distilled water. By changing the position of the electrode its distance from the specimen may be controlled. DC voltage ranging from 0 to 40 V is applied across the electrodes with a DC power supply (Advice Electronics Ltd., Rosh Ha-ayen, Israel) at an electrical field strength of up to 40 V/cm. The voltage and current data are recorded on an NI 5102 dual-channel 20 MS/s digitizer (National Instruments, Austin, Tex.) using a 10× high-voltage probe (Tetronix Inc., Beaverton, Oreg.). Voltage and current through samples are measured using a MultiLog™ 720 true RMS multimeter (Extech Instruments Co., Waltham, Mass.).

### SEM and Image Studies

To study the dry gels' structure and changes therein as a result of the electrical treatment, scanning electron microscopy (SEM) is performed. The dry gels (agarose and alginate) are taken from the same batches that produce samples for porosity and mechanical determinations. SEM micrographs are obtained for the external and internal features: for the former, the specimen is taken as is; for the internal features, a dry cellular solid is cut through with a double-edged razor blade to expose the internal surface features. Single downward cuts are used to produce a 1-mm thick slice. A 1:1 mixture of colloidal graphite in isopropyl alcohol and Ducco household glue is used as a conductive mounting adhesive and the sample is mounted on 10×10 mm aluminum SEM stubs coated with approximately 50 nm Au/Pd (60:40 w/w) in a Polaron E5100 unit equipped with a Peltier cooling stage. Samples are examined by electron microscopy (Jeol JSM 35C SEM, Tokyo, Japan) in high-vacuum mode (10⁻³ mm Hg) at an accelerating voltage of 25 kV.

The electron micrographs are then scanned (Hewlett Packard scanner, version 3.02, model 5300C) and saved as bmp files. The scanned micrographs are analyzed using Image Pro Plus (version 3.0.01.00, Media Cybernetics, L.P.). This program determines the number of pores and their area, in pixels, and translates the measurements into metric units. All results, statistical and otherwise, are calculated and plotted with the Excel software package (Microsoft Corporation, Soft Art Inc.).

### Porosity of the Dried Gels

The porosity (P) of the dried gels is calculated as: P=(1-bulk density/solid density) (Rassis, Nussinovitch, and Saguy 1997). Bulk density is estimated by dividing the sample weight by its overall volume. The latter is measured by displacement with 150- to 200-μm-diameter glass beads (Sigma). Particle (solid) density is derived by dividing the sample weight by its particle volume, as determined by pycnometer (Multi-Pycnometer, Quantachrome, Syosset, N.Y.). Following these pycnometer measurements, the same samples are used for bulk density determinations with glass beads (Marabi, Jacobson, Livings, and Saguy 2004).

### Mechanical Tests

Samples are compressed to 95% deformation between parallel lubricated plates, at a deformation rate of 10 mm/min, with an Instron Universal Testing Machine (UTM), Model 5544 (Instron Co., Canton, Mass.). The UTM is interfaced to a computer. ‘Merlin’ software (Instron Co.) performs data acquisition and conversion of the UTM's continuous voltage vs. time output into digitized stress vs. engineering strain relationships:

σ=F/A₀ (1)

ε_{E}=ΔH/H₀ (2)

where σ=stress; δ_{E}=engineering strain; F=momentary force; ΔH=momentary deformation, H₀−H(t); and A₀ and H₀ are the cross-sectional area and height of the original specimen, respectively.

The cross-sectional area of a compressed solid sponge rarely expands to any significant extent (Gibson and Ashby 1988); thus the engineering and “true” stress can be treated as equal for all practical purposes (Swyngedau, Nussinovitch, Roy, Peleg, and Huang 1991). Young's modulus was calculated as the slope of the initial linear portion of the stress vs. strain curve (Gibson and Ashby 1988).

### Statistical Analyses

In general, all statistical analyses are conducted with JMP software (SAS Institute 1995), including ANOVA and the Tukey-Kramer Honestly Significant Difference Method for comparisons of means.

The results suggested that only the combination of increased pH, gel type and matrix, and excess ions within the gel causes this phenomenon of surface enlargement and increased porosity. The described changes in structure and porosity could be potentially beneficial for various applications. The higher porosity of these gels could be advantageous in fields such as water denitrification, drug delivery and biological control of soil-borne root diseases. The freeze-dried hydrocolloid gels could also be useful as carriers for many food snacks, non-food matrices and biotechnological operations.

The following examples are intended to be merely illustrative in nature and to be construed in a non-limitative fashion.

### EXAMPLES

### Example 1

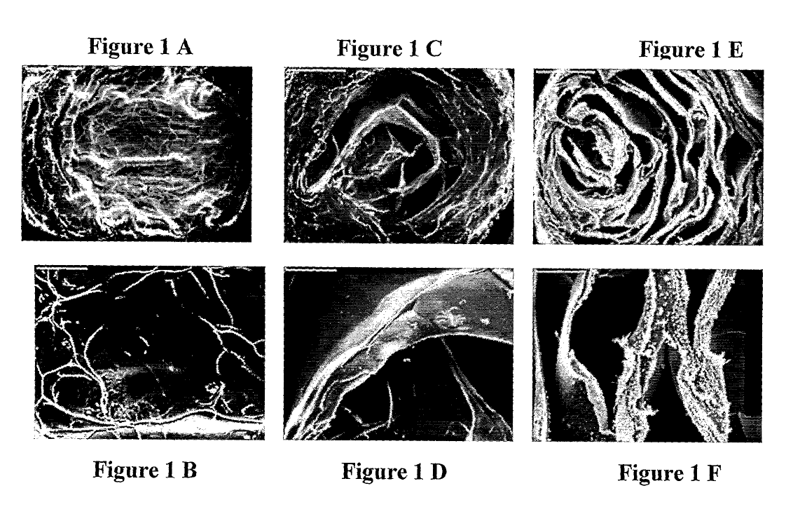

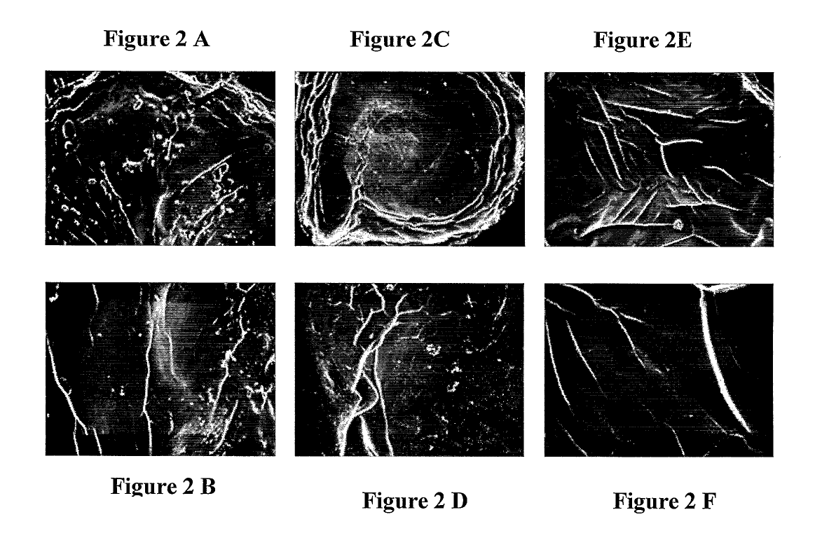

The application of a low electrical and freeze dehydration to hydrocolloid gels produced pores at the surface of the treated gel, which could change its release properties for special applications. Agarose appeared to be less affected by the DC electrical application (some small changes at the surface) than the alginate gel beads; in both gels, however, the shape of the affected area of the treated specimen resembled the shape of the anode (FIGS. 1 and 2). The freeze-dried alginate specimens' structure was more affected by the electrical treatment than that of agarose (i.e. more spaces and huge pores are observed versus almost no pores for the agarose).

### Example 2

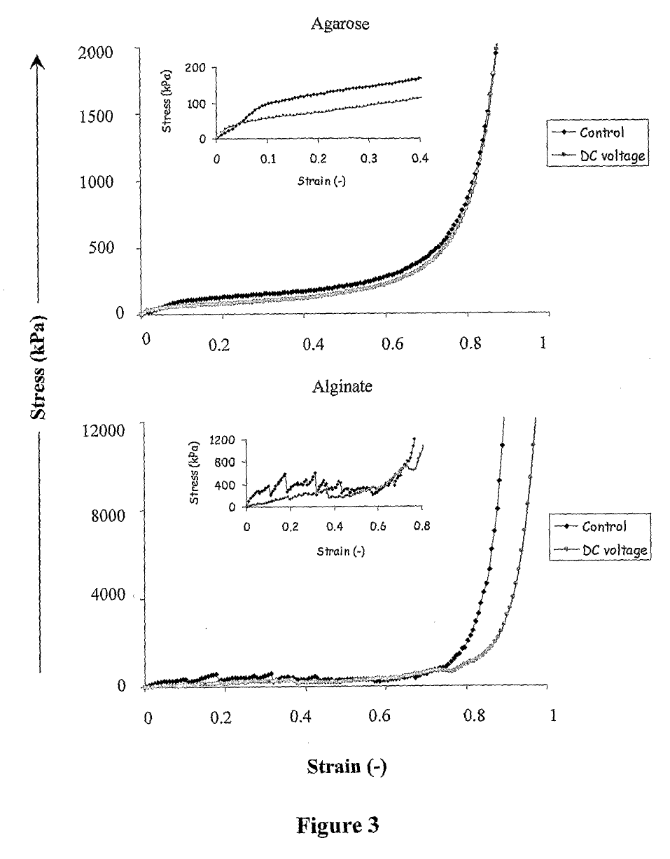

Analysis of the mechanical properties of these dried gels revealed the same trend, i.e. alginate was more influenced than agarose (FIG. 3). In both cases, the electrical treatment resulted in a weaker sponge (both insets of FIG. 3). For the electrified dried alginate gels, the stress-strain relationship was smoother than with the blank, i.e. the moiety was less brittle. Thus, this method can be used to change the texture of the dried gels (more or less crunchy), when desired (Nussinovitch, Corradini, Normand, and Peleg, 2000, (Nussinovitch, Corradini, Normand, and Peleg 2000; Nussinovitch, Corradini, Normand, and Peleg 2001). The curves of the control and electrically treated agarose sponges were nearly identical, while the alginate gel was effected by electrical treatment.

### Example 3

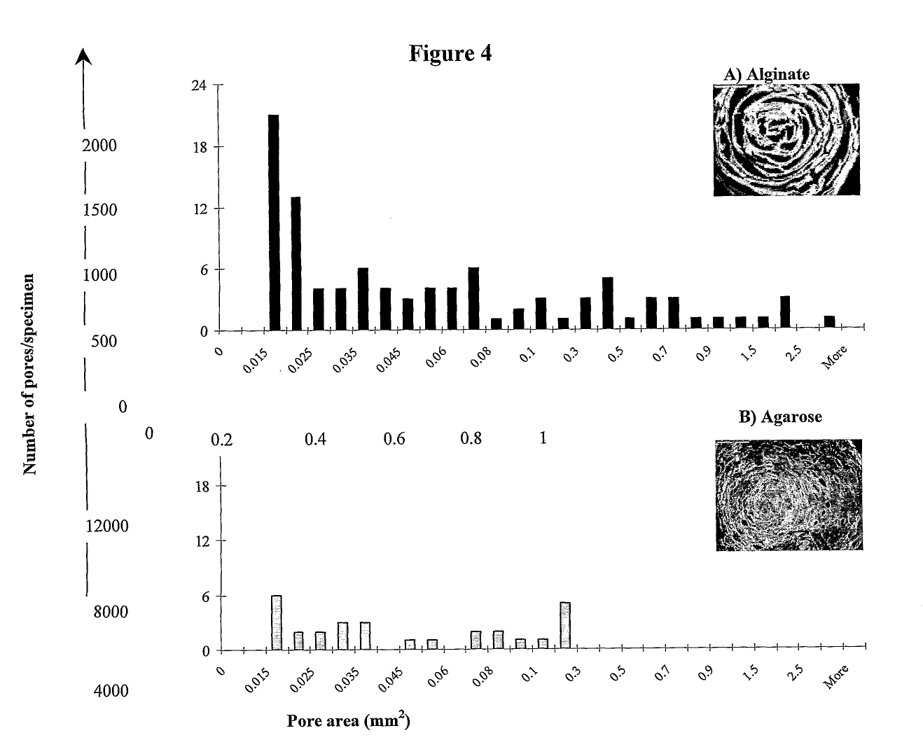

Additionally, an unexpected phenomenon was observed for the alginate sponges at the cathode end (FIGS. 1E and 2E): open spaces were created in a concentric pattern, resulting in a major increase in surface area. The increase in pore size and number could also be detected by image analysis (FIG. 4A, B). Whereas for the control (b) only a few small pores, up to 0.25 mm², were observed, in the electrically treated gels (a), more pores were observed, each one reaching 2.5 mm².

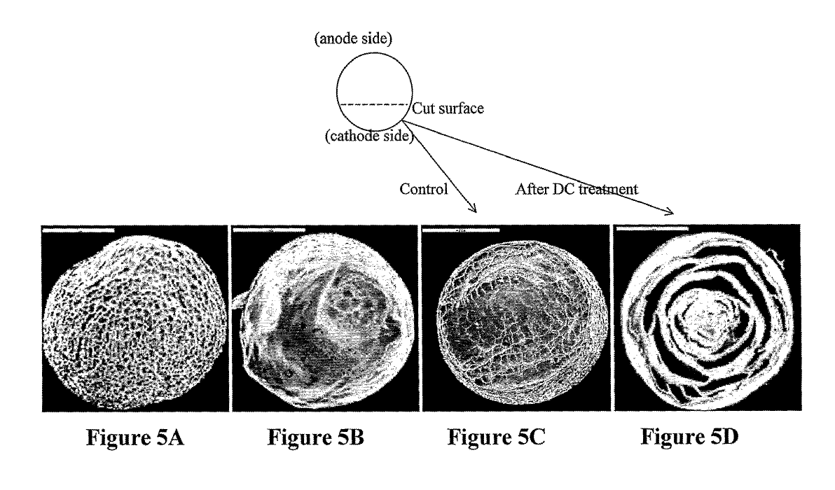

This phenomenon occurred at different alginate concentrations (1, 2 and 3%) and different gel sizes (3 mm and 20 mm in diameter; 2 and 12 mm in height). This phenomenon did not occur for alginate beads (4×4 mm, thickess by diameter). It may be that this phenomenon only occurs at the cut surfaces of alginate gels, where a less constricted network is present. As previously reported (Smidsrod and Skjak-Braek 1990), a spontaneous cross-linking reaction occurs at the gel surface, followed by a process that depends on the rate of calcium-ion diffusion into the formed bead, overcoming the resistance of the formed calcium-alginate layers that become further contracted with time. Consequently, a more constricted network is created on the gel bead's surface than at its core (Smidsrod and Skjal-Braek 1990). To verify this assumption, the electrical treatment was applied after cutting the surface (on the cathode side) (FIG. 5). It was confirmed that the phenomenon occurred after cutting a thin layer from the bead (FIG. 5D), and did not occur with the uncut bead (FIG. 5B).

### Example 4

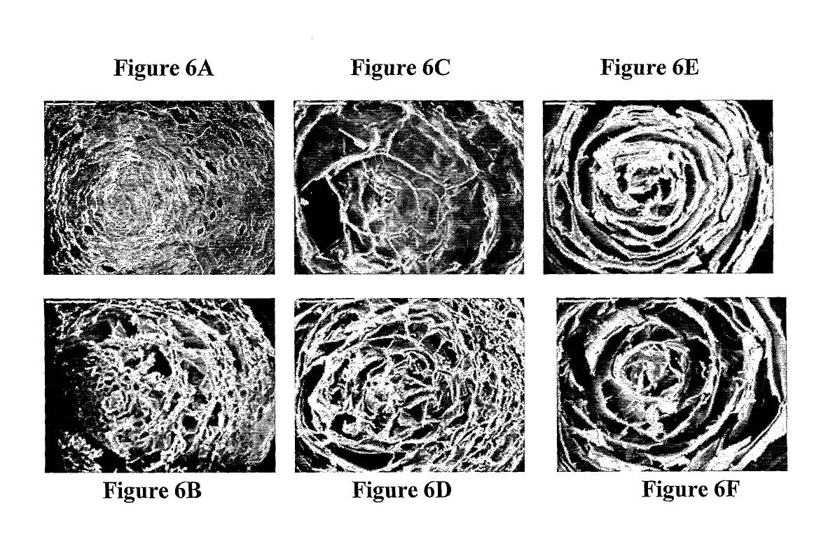

Although the outside surfaces of the untreated and electrically treated alginate specimens looked different, their inner structures were similar (FIG. 6). The pattern of circles (spaces) was observed in both cases and was related to the cross-linking direction, i.e., from the surface (outside of the bead) to the center. The similar structures could explain the small difference in total porosity observed for the electrically treated specimens (97%) vs. their untreated counterparts (95%) of the gel.

### Example 5

Recently there has been evidence of the important role of ion migration and the development of pH gradients in the reversible collapse of ionic gels in an electrical field. Such pH gradients were reported for agarose and alginate gels, as well as for plant tissues (Zvitov and Nussinovitch 2005). Higher pH values near the cathode and lower values near the anode have also been reported for different gel types (Kishi, Hasebe, Hara, and Osada 1990; Hirose, Giannetti, Marquardt, and Tanaka 1992; Ramanathan and Block 2001).

Alginate is a polyelectrolyte gel containing calcium ions (or other cations) as the cross-linking agent, whereas agarose is essentially a sulfate-free, neutral polysaccharide. It has been reported that for non-ionic gels that consist of H⁺ and OH⁻, no pH gradient is expected, because the conditions of electro-neutrality and the dissociation of water cannot be satisfied simultaneously; however, if the non-ionic gel contains ion impurities (such as in the agarose used in this study), then they will cause a pH gradient to form, depending on their concentration (Hirose, Giannetti, Marquardt, and Tanaka 1992). The pH gradient produced in the electrically treated agarose gels was less steep than in the alginate ones; this could explain why the agarose gels were less influenced by the electrical field.

### Example 6

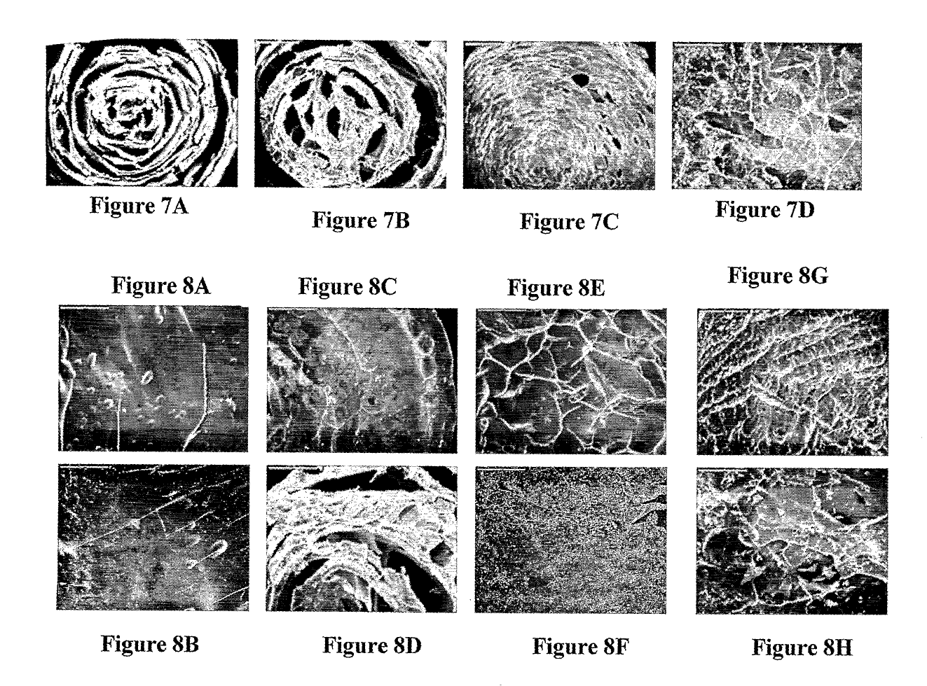

The pH gradient through the alginate gels yielded values of ca. 2 near the anode and ca. 12 near the cathode. It was important to check whether the phenomenon observed for the alginate gels at the cathode end was a result of the pH increase caused by the electrical field application. To study this question, a series of experiments were conducted. Agarose cylinders were immersed in a CaCl₂ solution prior to the electrification: these gels yielded similar pH values after the electrical treatment but did not exhibit surface pores after the freeze-dehydration (FIG. 7D). In addition, alginate cylinders were immersed in an alkaline solution (NaOH, pH 12) and were analyzed by SEM after freeze-dehydration (FIG. 7). The alkaline treatment produced a phenomenon similar to that observed at the surface of the electrically treated specimen. From these experiments, it became clear that pH has a major effect on structure, but is not the only influential factor.

### Example 7

Another possible explanation for the phenomenon is that alginate is a negatively charged polyelectrolyte relative to agarose, the latter being an essentially sulfate-free, neutral polysaccharide. To check this issue, a different polyelectrolyte gel, gellan, was examined and it was found that the phenomenon does not occur (FIG. 8E). This indicated that the reason behind the creation of such a structure is not solely the presence of a charged network. Furthermore, cold-set alginate also did not produce the pores observed for the spontaneously cross-linked alginate (FIG. 8F).

### Example 8

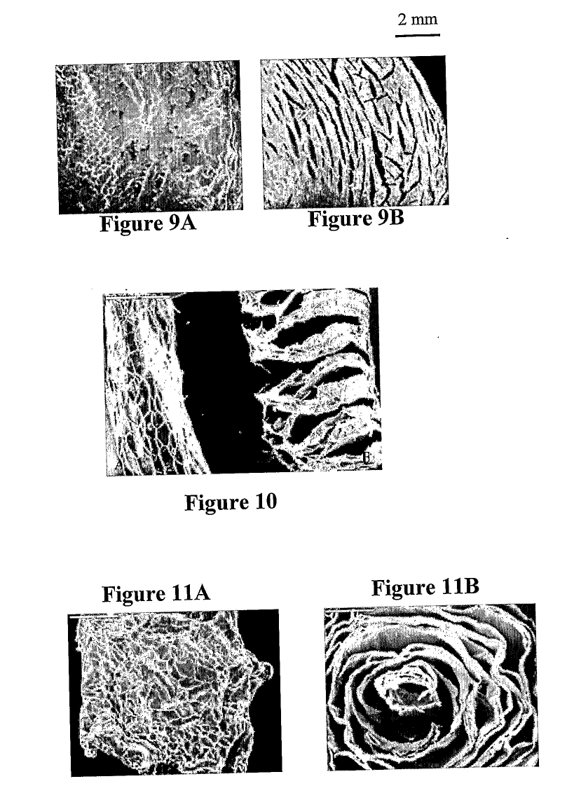

It was hypothesized that the phenomenon of concentric open spaces is related to the cross-linking pattern followed during production of the alginate gel. Thus, it should occur with other polyanions that gel via a cross-linking mechanism. To verify this assumption, low-methoxy-pectin (LMP) gels were produced by the same procedure used for the alginate cylinders (see Materials and Methods). Indeed, a similar phenomenon was observed for the LMP gel (FIG. 9). The cross-linking in both the alginate and the LMP was concentric, due to the process of their formation. To achieve a different pattern and verify that the observed phenomenon is related to the cross-linking pattern, a different type of cross-linking was obtained by placing the alginate solution in a prismatic cellulose-acetate receptacle which allowed the calcium ions to diffuse only from the top, thus creating a different cross-linking pattern (FIG. 10). The alginate gel specimen was electrically treated and the phenomenon of increased surface area occurred after freeze-dehydration, but this time not concentrically but in layers, in accordance with the cross-linking pattern, as expected.

### Example 9

Another issue was the importance of the counter ions in the gel. These results showed that the presence of excess ions is not sufficient to create these surface changes, i.e., that the phenomenon of concentric open spaces [agarose immersed in CaCl₂ did not exhibit them]. However, although the counter ions are not the only factor leading to the formation of these pores, they do play an important role. Alginate gel cylinders, which were immersed prior to the electrical treatment in distilled water until excess ions diffused out, did not have surface pores after the electrical treatment and freeze-dehydration (FIG. 8B); moreover, after immersion in an alkali solution and freeze-dehydration, these gels did not produce this phenomenon either. On the other hand, alginate gel cylinders which were cross-linked with BaCl₂ yielded a similar structure (FIG. 8D), showing that the phenomenon is not restricted to a particular cross-linking agent.

### Example 10

The phenomenon of concentric open spaces occurred during freezing and was not due to freeze-dehydration. FIG. 11 shows the alginate cylinder after freezing and thawing (and air-drying). It is clear that the phenomenon of concentric open spaces occurred during the freezing, and it is also important to note that the phenomenon was observed immediately after removal from the freezer.

### Example 11

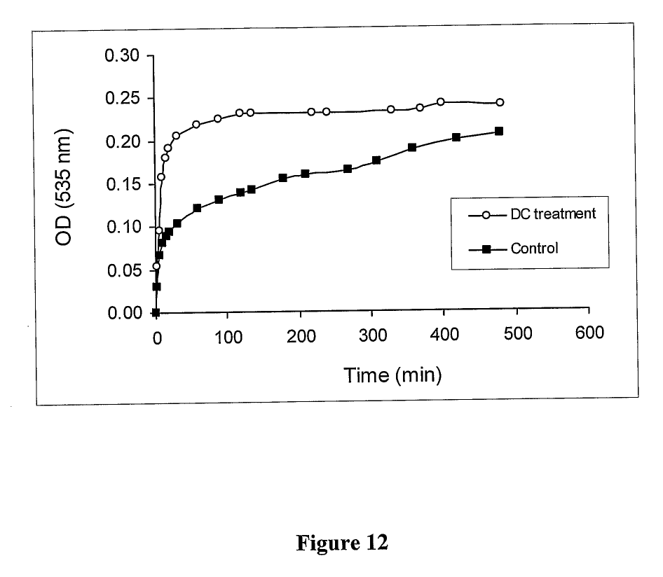

The release of pigment (i.e. betanin) from a dried alginate specimen following electrical treatment was measured. Untreated and electrically treated (10 V/cm for 1 min) freeze-dried alginate specimens were immersed in a 1% (w/w) betanin solution for 24 h; after dehydration in air, the gel specimens were rehydrated in water to examine the diffusion of the betanin from these specimens versus time. Betanin diffusion was evaluated by spectrophotometric absorption measurements in a Milton Roy Spectronic 601 spectrophotometer (Spectronic Unicarn, N.Y.) at 535 nm. A higher and more rapid increase in the immersion solution's absorbance was observed with the electrically treated specimen than with its untreated counterpart as shown in FIG. 12. This indicates that electrification of freeze-dried gels according to the method of the present invention results in increased release of substances form the treated gel.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

1. Gibson L J, Ashby M F (1988) Cellular Solids: Structure and Properties. Pergamon Press, Oxford,
2. Hirose Y, Giannetti G, Marquardt J, Tanaka T (1992) Migration of Ions and Ph Gradients in Gels Under Stationary Electric-Fields. Journal of the Physical Society of Japan 61: 4085-4097
3. K ishi R, Hasebe M, Hara M, Osada Y (1990) Mechanism and Process of Chemomechanical Contraction of polyelectrolyte Gels Under Electric Field. Polymers for Advanced Technologies 1: 19-25
4. M arabi A, Jacobson M, Livings S J, Saguy I S (2004) Effect of mixing and viscosity on rehydration of dry food particulates. European Food Research and Technology 218: 339-344
5. Nussinovitch A, Corradini M G, Normand M D, Peleg M (2000) Effect of sucrose on the mechanical and acoustic properties of freeze-dried agar, kappa-carrageenan and gellan gels. Journal of Texture Studies 31: 205-223
6. Nussinovi tch A, Corradini M G, Normand M D, Peleg M (2001) Effect of starch, sucrose and their combinations on the mechanical and acoustic properties of freeze-dried alginate gels. Food Research International 34: 871-878
7. Nussinovitch A, Peleg N, MeyTal E (1996b) Apparatus for the continuous monitoring of changes in shrinking gels. Food Hydrocolloids 10: 137-141
8. Nussinovitch A, Peleg N, MeyTal E (1996a) Apparatus for the continuous monitoring of changes in shrinking gels. Food Hydrocolloids 10: 137-141
9. R amanathan S, Block L H (2001) The use of chitosan gels as matrices for electrically-modulated drug delivery. Journal of Controlled Release 70: 109-123
10. Rassis D, Nussinovitch A, Saguy I S (1997) Tailor-made porous solid foods. International Journal of Food Science and Technology 32: 271-278
11. S AS Institute. SAS Institute JMP statistics and graphics guide. Version 3.1.1995. Cary, N.C., SAS Institute Inc. (Computer Program)
12. S midsrod O, Skjak-Braek G (1990) Alginate as Immobilization Matrix for Cells. Trends Biotechnol 71-78
13. S wyngedau S, Nussinovitch A, Roy I, Peleg M, Huang V (1991) Comparison of 4 Models for the Compressibility of Breads and Plastic Foams. Journal of Food Science 56: 756-759
14. Tal Y, van Rijn J, Nussinovitch A (1999) Improvement of mechanical and biological properties of freeze-dried denitrifying alginate beads by using starch as a filler and carbon source. Applied Microbiology and Biotechnology 51: 773-779
15. Tanaka T, Nishio I, Sun S, Uneo-Nishio S (1982) Collapse of gels in an electric field. Science 218: 467-469
16. Z vitov R, Nussinovitch A (2001) Weight, mechanical and structural changes induced in alginate gel beads by DC electrical field. Food Hydrocolloids 15: 33-42
17. Z vitov R, Nussinovitch A (2003) Changes induced by DC electrical field in agar, agarose, alginate and gellan gel beads. Food Hydrocolloids 17: 255-263
18. Z vitov R, Nussinovitch A (2005) Low DC electrification of gel-plant tissue ‘sandwiches’ facilitates extraction and separation of substances from Beta vulgaris beetroots. Food Hydrocolloids 19: 997-1004
19. Z vitov R, Schwartz A, Zamski E, Nussinovitch A (2003) Direct current electrical field effects on intact plant organs. Biotechnology Progress 19: 965-971
20. Z vitov R, Zohar-Perez C, Nussinovitch A (2004) Short-duration low-direct-current electrical field treatment is a practical tool for considerably reducing counts of gram-negative bacteria entrapped in gel beads. Applied and Environmental Microbiology 70: 3781-3784

1.-38. (canceled)

39. An electrified, freeze-dried hydrocolloid gel having a structure comprising concentric layers of hydrocolloid gel material, separated by intervening spaces.

40. The gel of claim 39 having at least one improved property selected from the group consisting of: porosity, density of pores, size of pores, volume, surface area ratio, surface area per volume, strength, elasticity, swelling ability, proclivity to decomposition and the ability to remain intact under different conditions, compared to hydrocolloid gel not subjected to combined electrification and freeze-drying, or to hydrocolloid gel not having concentric layers.

41. The gel of claim 39 in the form of beads, plates, strips, sheets or cylinders.

42. The gel of claim 39 wherein the hydrocolloid is selected from the group consisting of alginate, agar, agarose, pectin, carrageenan, and low methoxy pectin.

43. The gel of claim 39 wherein the concentric layers appear generally parallel to the circumference of the gel.

44. The gel of claim 39 further comprising at least one active agent selected from the group consisting of a chemical agent, a biological agent, an agriculturally active agent, and a medicinally active agent.

45. The gel of claim 44 wherein the medicinally active agent is a drug, a pro-drug, a combination of drugs, a diagnostic agent and an imaging agent useful in therapy or diagnosis, or wherein the agriculturally active agent is selected from an agro-chemical compound used for control of pests, a fertilizer and a biological compound.

46. A therapeutic composition that includes an electrified, freeze-dried hydrocolloid gel according to claim 39 that has an increased surface area per volume.

47. An agricultural agent for biological control of plant diseases, wherein the agent includes an electrified, freeze-dried hydrocolloid gel according to claim 39 that has an increased surface area per volume.

48. A biological composition for drug delivery, for decomposing toxic substances, or for use in biotechnological processes or in the food industry, comprising an electrified freeze-dried hydrocolloid gel according to claim 39 that has an increased surface area per volume.

49. The biological composition according to claim 48 wherein the biotechnological process is selected from entrapping ingredients, entrapping microorganisms, or use as a mini-reactor.

50. A method for preparing an electrified freeze dried hydrocolloid gel having modified properties, which comprises:
providing a gel specimen;
electrifying the gel specimen by applying a DC voltage;
freezing the gel specimen; and
freeze-dehydrating the gel,
thereby changing at least one of the properties of the gel.

51. The method of claim 50 wherein the electrified freeze-dried gel comprises concentric layers of gel separated by intervening spaces.

52. The method of claim 50 wherein the electrified freeze-dried gel has increased surface area per volume compared to a gel of the same composition that has not been subjected to electrifying and freezing.

53. The method of claim 50 wherein the change in at least one of the properties is increased porosity or a change in gel texture.

54. The method of claim 50 wherein the DC voltage applied when electrifying ranges from 0.1-40 V at electrical field strength up to 40 V/cm.

55. The method of claim 50 wherein the electrifying creates concentric layers at the cathode end of the gel.

56. The method of claim 50 which further comprises, prior to electrifying, adding an ion solution to the gel, or modifying gel pH.

57. The method of claim 56 wherein the ion solution is CaCl2 or BaCl2.

58. A pharmaceutical composition comprising an electrified, freeze-dried hydrocolloid gel according to claim 39, at least one therapeutic agent, and optionally a pharmaceutically acceptable carrier or excipient.

59. The pharmaceutical composition according to claim 58 wherein the pharmaceutical composition is dry, fluid, or semi-fluid.
